(19) United States
(12) Patent Application Publication
(10) Pub. No.: US 2015/0197644 A1
(43) Pub. Date: Jul. 16, 2015

(54) Anti-Biofouling Networks And Applications thereof

(71) Applicant: Chung Yuan Christian University, Tao-Yuan (TW)

(72) Inventors: Yung Chang, Tao-Yuan (TW); Hui-Shan Yang, Tao-Yuan (TW); Kueir-Rarn Lee, Tao-Yuan (TW); Juin-Yih Lai, Tao-Yuan (TW)

(21) Appl. No.: 14/661,332

(22) Filed: Mar. 18, 2015

Related U.S. Application Data
(63) Continuation-in-part of application No. 13/690,202, filed on Nov. 30, 2012.

(30) Foreign Application Priority Data
Nov. 29, 2012 (TW) 101144643

(51) Int. Cl. C09D 5/16; A61L 29/08; A61L 27/50; A61L 31/14; A61L 29/14; A61L 31/10; A61L 27/34

(52) U.S. Cl. CPC C09D 5/1637; A61L 31/10; A61L 29/085; A61L 27/34; A61L 31/14; A61L 29/14; A61L 27/50; A61L 2420/06

(57) ABSTRACT

The present invention provides a biomimetic agent for anti-biofouling networks and application thereof. The antibiofouling networks have a mole mass ratio of poly(vinylpyrolidone) to poly(sulfobetaine methacrylate) in a range of 70/30˜42/58 and are derived from a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate).

### CROSS REFERENCE TO RELATED APPLICATIONS

This application is a Continuation In Part of applicant's earlier application Ser. No. 13/690,202, filed Nov. 30, 2012.

### BACKGROUND OF THE INVENTION

1. Field of the Invention

The present invention is generally related to a biomimetic agent, and more particularly to a biomimetic agent for anti-biofouling networks and application thereof.

2. Description of the Prior Art

At present, anti-biofouling surface treatment is one of the important techniques in various applications. An anti-biofouling surface indicates a biomolecule adhering resistant surface where the biomolecule is, for example, protein, blood, cells, bacteria, etc. The coating agent used in anti-biofouling surface treatment is generally considered to make a surface become hydrophilic in assisting of anti-biofouling according to prior arts, but the anti-biofouling effect in practice is not enough.

Furthermore, it have been reported that zwitterionic molecules or polymers are used as the anti-biofouling agent. For example, Wu et al. (L. Wu, J. Jasinski, S. Krishnan, J. Appl. Polymer Scienec, Vol. 124, 2154(2012)) disclosed a block copolymer comprising a betaine moiety, sulfobetaine moiety or carboxybetaine moiety as an anti-biofouling agent and anti-biofouling surface treatment by spin-coating the anti-biofouling agent. However, from FIG. 8 of the report, the experiment of the protein adsorption effect shows that the anti-biofouling agent does not have 80% Y of protein adsorption resistance and thus is not an excellent anti-biofouling agent having less than 20% of protein adsorption. Besides, the anti-biofouling effect also includes other molecules such as blood, cells, bacteria resistance and these properties were not discussed in the report.

According to prior arts, although in general a surface being hydrophilic or having zwitterionic moieties shows anti-biofouling, an excellent anti-biofouling surface to be useful should be treated by a novel anti-biofouling agent comprising a specific structure.

### SUMMARY OF THE INVENTION

In accordance with the present invention, a biomimetic agent for anti-biofouling coating and a method for making the same are provided. The present invention utilizes a polymer comprising a specific structure and zwitterionic or pseudo-zwitterionic moieties as an effective component of anti-biofouling to achieve anti-biofouling effect.

One objective of the present invention is to provide a biomimetic agent for anti-biofouling coating to apply to various surfaces, such as polymeric, metallic, ceramic and porous surfaces, by a simple well-known coating method, such as dipping, spin-coating and so forth.

One objective of the present invention is to provide a method for making a biomimetic agent for anti-biofouling coating to use a simple well-known coating method, such as dipping, spin-coating and so forth, to be easily applied to surfaces with a large area and porous surfaces. Therefore, mass-production can be easily implemented and no special or large-scaled equipment is required to reduce production cost.

Another objective of the present invention is to provide a method for preventing from fouling of a biomolecule on a substrate, the method comprising applying a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate); and performing a thermal process at 120° C. above to have the copolymer to form a network on the substrate surface so as to prevent from fouling of a biomolecule on the substrate.

Still another objective of the present invention is to provide a antibiofouling network, the antibiofouling network has the mole mass ratio of poly(vinylpyrolidone) to poly(sulfobetaine methacrylate) in a range of 70/30˜42/58 and is derived from a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate) by heating the copolymer at 120° C. above.

Accordingly, the present invention discloses a biomimetic agent for anti-biofouling coating, comprising a block, random or alternating copolymer having a general formula (1): AUₙBUₘ wherein AU represents a repeating unit comprising an anchoring moiety, being a methylene moiety with substituents R¹ and R² having a structure of formula (2): —CR¹R²—; BU represents a repeating unit comprising a zwitterionic moiety or a pseudo-zwitterionic moiety, being an ethylene moiety with a substituent R³ having a structure of formula (3): —CH₂CR³H— or a propylene moiety with substituents R⁴ and R⁵ having a structure of formula (4): —CR⁴HCH₂CR⁵H—; m is an integer of 5˜120; n is an integer of 5˜120; R¹ represents a C3˜18 chained, branched or cyclic alkyl moiety, ester moiety (i.e. —COOR^{x}, where R^{x} represents a C3˜18 chained, branched or cyclic alkyl moiety, aryl moiety having substituent(s) of C1˜18 alkyl, or C5˜12 heteroaryl moiety), phenyl moiety having substituent(s) of C1˜18 alkyl, or C5˜12 heteroaryl moiety; R² represents a hydrogen atom (H) or methyl moiety (CH₃); R³ represents —COOR′ or —CONR″H; R⁵ represents —COOR′ or —CONR″H when R⁴ represents a hydrogen atom or R⁵ represents a cationic moiety when R⁴ represents a carboxylic moiety; R′ and R″ individually represent a betaine moiety, sulfobetaine moiety or carboxybetaine moiety.

Furthermore, the present invention discloses a method for making a biomimetic agent for anti-biofouling coating, comprising the following steps: providing a monomer comprising an anchoring moiety; providing a monomer comprising a zwitterionic moiety or a pseudo-zwitterionic moiety; and performing atomic transfer radical polymerization under existence of a catalyst and a polymerization initiator in nitrogen environment to have the monomer comprising an anchoring moiety and the monomer comprising a zwitterionic moiety or a pseudo-zwitterionic moiety react to form a block, random or alternating copolymer having a general formula (1): AUₙBUₘ.

Moreover, the present invention discloses a coating composition for anti-biofouling, comprising: the above mentioned biomimetic agent and a solvent.

In addition, the present invention discloses a method for preventing from fouling of a biomolecule on a substrate, the method comprising: applying a copolymer containing a poly(vinylpyrolidone) and a zwitterionic moieties and performing a thermal process at 120° C. above to have the copolymer to form a network on the substrate so as to prevent from fouling of a biomolecule on the substrate. The biomolecules comprises fibrinogen, platelets, erythrocytes, fibroblast, and E. coli.

Finally, the present invention discloses an antibiofouling network which has the mole mass ratio of poly(vinylpyrolidone) to poly(sulfobetaine methacrylate) in a range of 70/30˜42/58 and is derived from a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate) by heating the copolymer at 120° C. above.

According to the biomimetic agent for anti-biofouling coating and networks and both of the method for making the same of the present invention and application, a polymer comprising a specific structure and zwitterionic or pseudo-zwitterionic moieties is used as an effective component of anti-biofouling to achieve excellent anti-biofouling effect. Besides, the biomimetic agent can be applied to surfaces with a large area and porous surfaces easily by a simple well-known coating method, such as dipping, spin-coating and so forth. Therefore, mass-production can be easily implemented and no special or large-scaled equipment is required to reduce production cost.

### BRIEF DESCRIPTION OF THE DRAWINGS

FIG. 1 shows FT-IR spectra of the PVP (V100-S0), poly(VP-co-SBMA) (V75-S25, V50-S50, V37-S63, V25-S75), and PSBMA (V0-S100) gels without thermo-setting;

FIG. 2(a) to FIG. 2(b) show XPS analysis of the PVP (V100-S0), poly(VP-co-SBMA) (V75-S25, V50-S50, V37-S63, V25-S75), and PSBMA (V0-S100) (co)polymer (a) gels without thermo-setting and (b) networks with thermo-setting;

FIG. 3 shows Human fibrinogen adsorption and diiodomethane contact angles of the PVP (V100-S0), poly(VP-co-SBMA) (V75-S25, V50-S50, V37-S63, V25-S75), and PSBMA (V0-S100) (co)polymer gels without thermo-setting and networks with thermo-setting, using tissue culture polystyrene (TCPS) as positive control. (* indicating V100-S0 and V75-S25 networks cracked after the thermo-setting);

FIG. 4(a) to FIG. 4(b) show CLSM images of (a) human platelets adhesion and (b) erythrocytes attachment observed on the PVP (V100-S0), poly(VP-co-SBMA) (V75-S25, V50-S50, V37-S63, V25-S75), and PSBMA (V0-S100) (co)polymer gels without thermo-setting and networks with thermo-setting. All images are at a magnification of 1000;

FIG. 5 shows Hemolysis of RBC solution in the presence of the PVP (V100-S0), poly(VP-co-SBMA) (V75-S25, V50-S50, V37-S63, V25-S75), and PSBMA (V0-S100) (co)polymer gels without thermo-setting and networks with thermo-setting, using tissue culture polystyrene (TCPS) as positive control. (* indicating V100-S0 and V75-S25 networks cracked after the thermo-setting);

FIG. 6 shows Statistical analysis of HT1080 cell attachment on the surfaces of the PVP (V100-S0), poly(VP-co-SBMA) (V75-S25, V50-S50, V37-S63, V25-S75), and PSBMA (V0-S100) (co)polymer gels without thermo-setting and networks with thermo-setting, using tissue culture polystyrene (TCPS) as positive control. Cell culture was performed at an initial concentration of 2×10⁴ cells/ml. (* indicating V100-S0 and V75-S25 networks cracked after the thermo-setting);

FIG. 7 shows Fluorescence microscopic images of E. coli attachment on the surfaces of the PVP (V100-S0), poly(VP-co-SBMA) (V75-S25, V50-S50, V37-S63, V25-S75), and PSBMA (V0-S100) (co)polymer gels without thermo-setting and networks with thermo-setting. All images are at a magnification of 1000×; and

FIG. 8(A)˜(D) show application of poly(VP-co-SBMA) coating on the metal stent, where (A) shows the stainless stain disk coated by copolymer gels and networks, (B) shows the results of fibrinogen adsorption test of copolymer gels/networks coated stainless steel disk, (C) shows the results of whole blood cell attachment test of copolymer gels/networks coated stainless steel disk and (D) shows the result of the stability of copolymer gels and networks coated on stainless steel disks in 30 days.

### DESCRIPTION OF THE PREFERRED EMBODIMENTS

What is probed into the invention is a biomimetic agent for anti-biofouling coating and networks. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The biomimetic agent for anti-biofouling coating according to the present invention can be coated on a surface to perform anti-biofouling surface treatment so as to achieve the effect of anti-biofouling. For example, the treated surface becomes resisting adherence of biomolecules like protein, blood, cell, bacteria, etc. The biomimetic agent according to the present invention is a compound having a structure imitating nature of a biomolecule. The biomimetic agent according to the present invention has a specific structure and zwitterionic or pseudo-zwitterionic moieties to possess the anti-biofouling characteristic. Besides, usually the coating agent usually has a problem of adhering to a treating surface. The coating agent according to the present invention uses a specific anchoring moiety not only to solve the adherence problem but also to provide excellent properties of coating, film-forming, and surface adherence.

In one embodiment of the invention, a biomimetic agent for anti-biofouling coating is disclosed. The biomimetic agent comprising a block, random or alternating copolymer having a general formula (1): AUₙBUₘ wherein AU represents a repeating unit comprising an anchoring moiety, being a methylene moiety with substituents R¹ and R² having a structure of formula (2): —CR¹R²—; BU represents a repeating unit comprising a zwitterionic moiety or a pseudo-zwitterionic moiety, being an ethylene moiety with a substituent R³ having a structure of formula (3): —CH₂CR³H— or a propylene moiety with substituents R⁴ and R⁵ having a structure of formula (4): —CR⁴HCH₂CR⁵H—; m is an integer of 5˜120; n is an integer of 5˜120.

Specifically, the biomimetic agent has the following structure shown by formula (A) or (B).

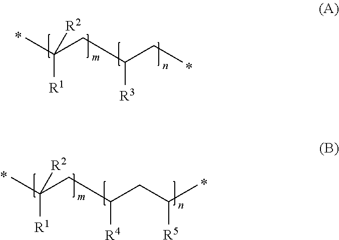

In the formula (A) and (B), R¹, R², R³, R⁴ and R⁵ represent the same moieties as the above.

R¹ represents a C3˜18 chained, branched or cyclic alkyl moiety, ester moiety (i.e. —COOR^{x}, where R^{x} represents a C3˜18 chained, branched or cyclic alkyl moiety, aryl moiety having substituent(s) of C1˜18 alkyl, or C5˜12 heteroaryl moiety), phenyl moiety having substituent(s) of C1˜18 alkyl, or C5˜12 heteroaryl moiety. “C3˜18”, “C1˜18” or “C5˜12” means the number of carbon atoms in the moiety. That is, for example, C3˜18 chained, branched or cyclic alkyl moiety means alkyl having three carbon atoms˜eighteen carbon atoms, such as propyl, isopropyl, butyl, isobutyl, pentyl, hexyl, heptyl, octyl, nonyl, undecyl, dodecyl, tridecyl, tetradecyl, pentadecyl, hexadecyl, heptadecyl, octadecyl, cyclopentyl, cyclohexyl, cycloheptyl, etc. The ester moiety (i.e. —COOR^{x}), for example, is phenyl, tolyl, benzyl, 2-pyrrolidone-1-yl, 2-pyridyl, phenoxycarbonyl (—C(═O)—OC₆H₅), benzyloxycarbonyl (—C(═O)—OCH₂C₆H₅) and N,N-diphenylamino-para-phenoxycarbonyl

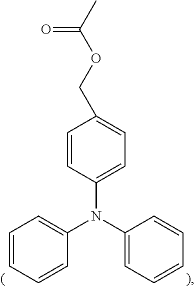 [SMILES: CC(=O)OCc1ccc(N(c2ccccc2)c2ccccc2)cc1]

etc. The aryl moiety, for example, is phenyl, tolyl, benzyl, etc. The C5˜12 heteroaryl moiety, for example, is 2-pyrrolidone-1-yl, 2-pyridyl, etc. The open dashed symbol “-” indicates the linkage bond and the following “-” also has the same meaning.

R² represents a hydrogen atom (H) or methyl moiety (CH₃). R³ represents —COOR′ or —CONR″H. R⁵ represents —COOR′ or —CONR″H when R⁴ represents a hydrogen atom or R⁵ represents a cationic moiety when R⁴ represents a carboxylic moiety. R′ and R″ individually represent a betaine moiety, sulfobetaine moiety or carboxybetaine moiety. Preferably, m is an integer of 10˜80; n is an integer of 10˜80. More preferably, m is an integer of 20˜40; n is an integer of 20˜40.

The cationic moiety represented by R⁵, for example, is N,N-dimethylammnio-ethylene-1-amino-vinyl (—C(═CH₂)NH₂CH₂CH₂N(CH₃)₂H), N,N-dimethylammnio-propylene-1-amino-vinyl (—C(═CH₂)NH₂CH₂CH₂CH₂N(CH₃)₂H), N,N-dimethylammnio-butylene-1-amino-vinyl (—C(═CH₂)NH₂CH₂CH₂CH₂CH₂N(CH₃)₂H), and N,N-dimethylammnio-pentylene-1-amino-vinyl (—C(═CH₂)NH₂CH₂CH₂CH₂CH₂CH₂N(CH₃)₂H).

Specifically, the biomimetic agent has the following structure shown by formula (A-1)˜(A-21).

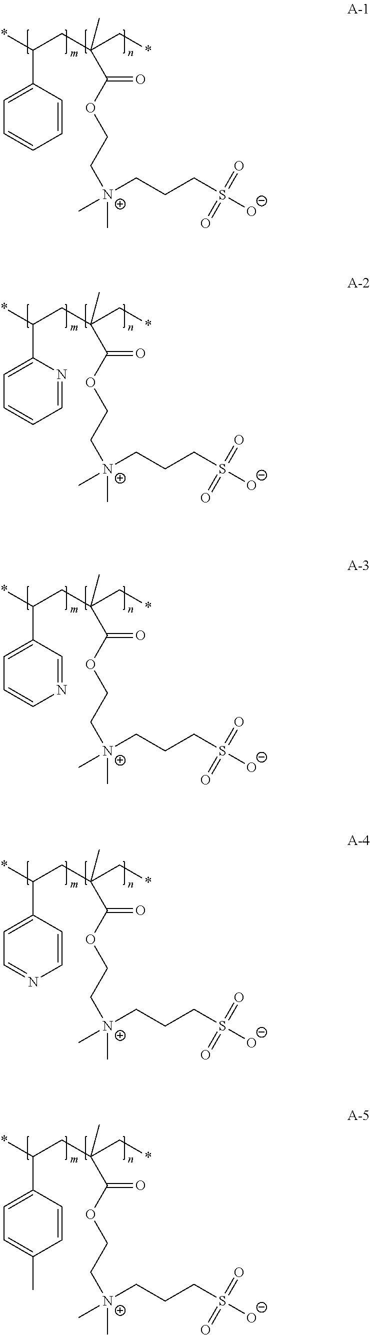

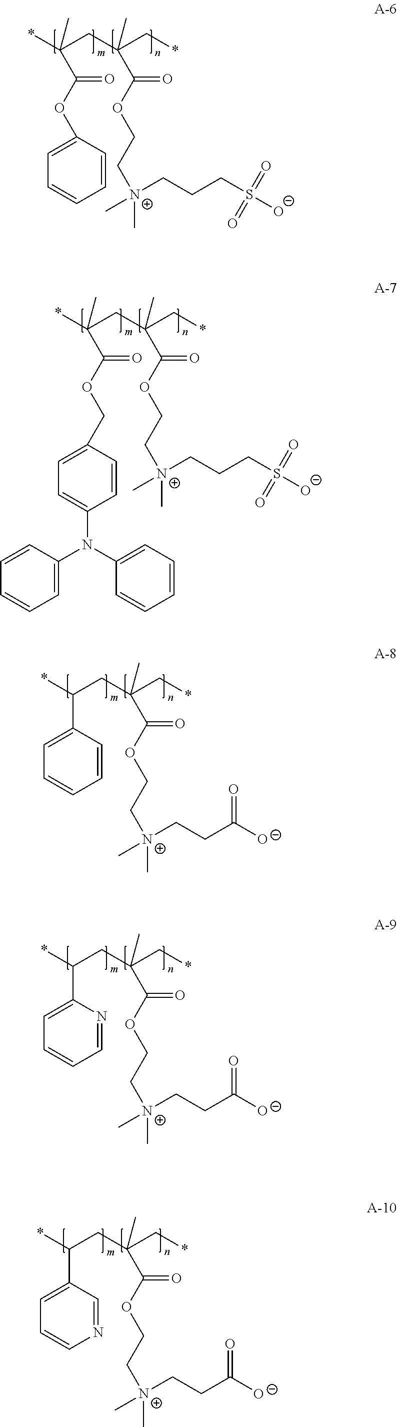

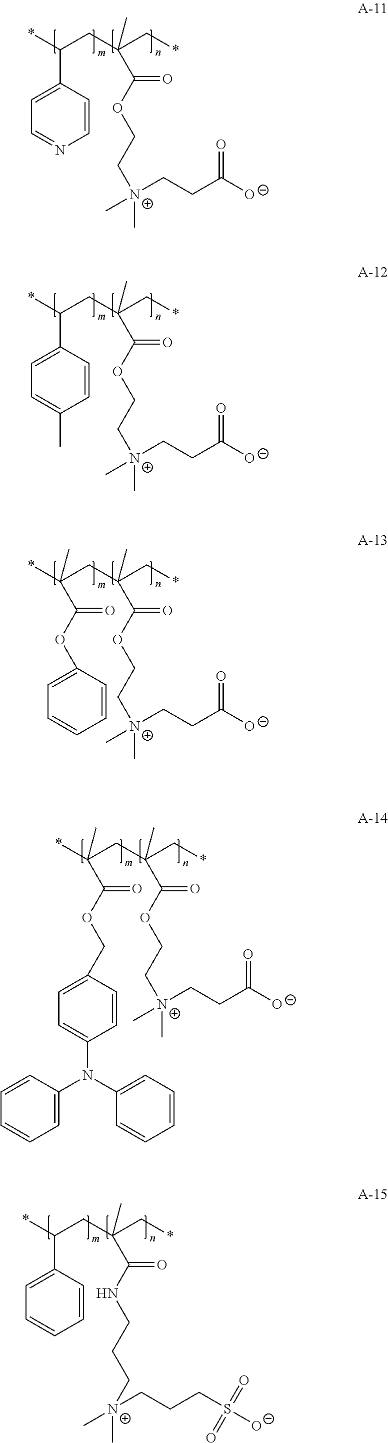

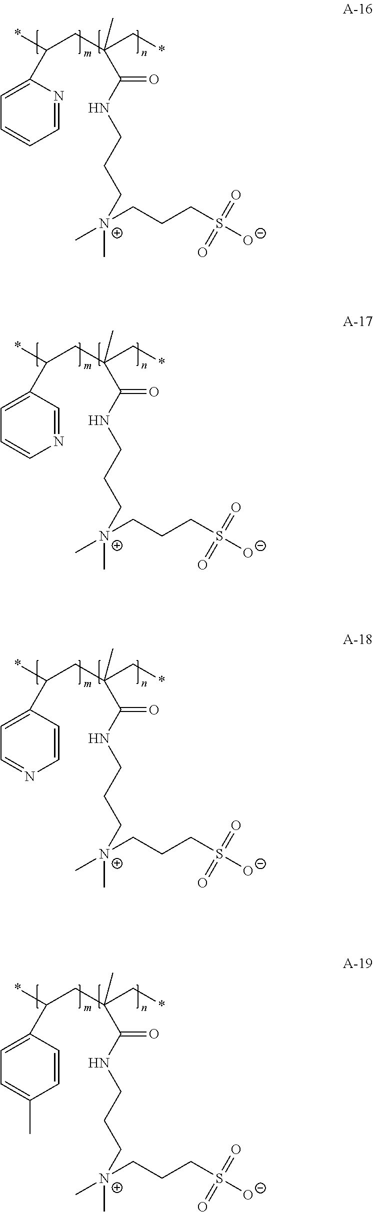

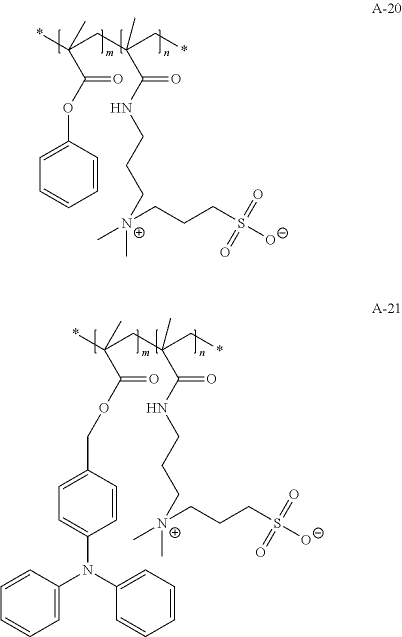

The above biomimetic agents with formula (A-1)˜(A-21) comprise rod-like anchoring moieties where m and n have the same meaning as the above. The biomimetic agents with formula (A-1)˜(A-21) can be a block, random or alternating copolymer.

Furthermore, the biomimetic agent has the following structure shown by formula (A-22)˜(A-33). The following biomimetic agents with formula (A-22)˜(A-33) comprise comb-like anchoring moieties where m and n have the same meaning as the above. The biomimetic agents with formula (A-22)˜(A-33) can be a block, random or alternating copolymer.

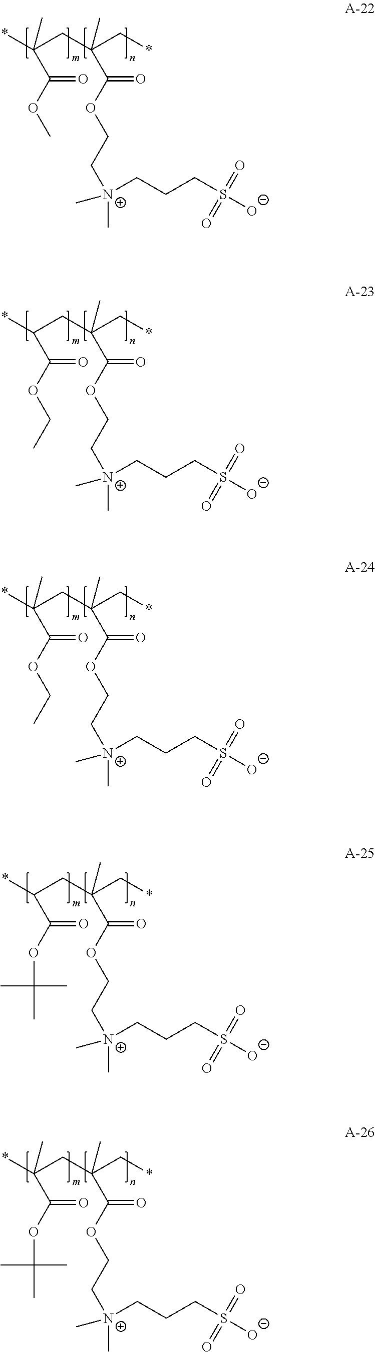

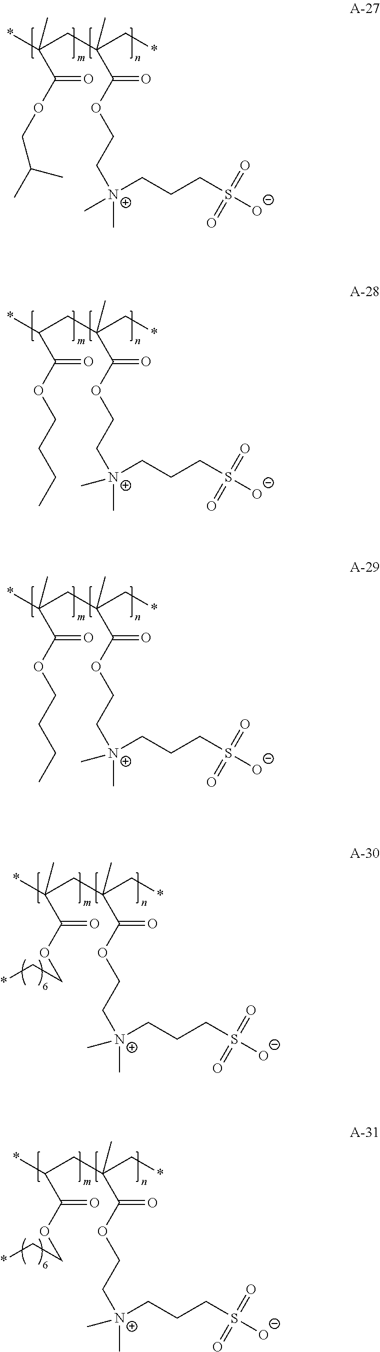

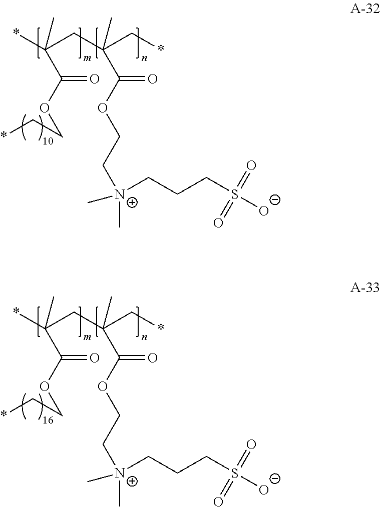

Furthermore, the biomimetic agent has the following structure shown by formula (B-1) or (B-2).

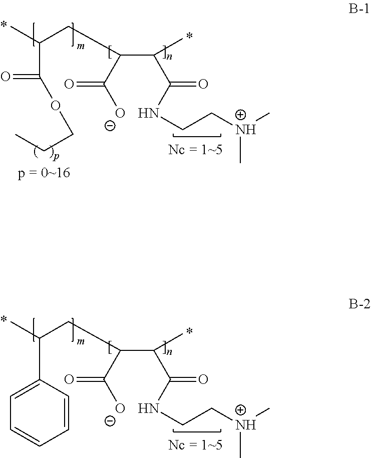

The biomimetic agents with formula (B-1)˜(B-2) can be an alternating copolymer.

Furthermore, the biomimetic agent has the following structure shown by formula (B-3).

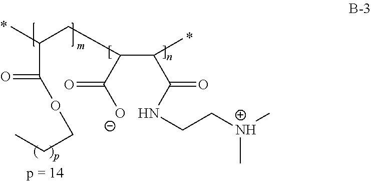

The biomimetic agents with formula (B-3) can be a block or random copolymer.

Furthermore, the biomimetic agent has the following structure shown by formula (C-1)˜(C-6).

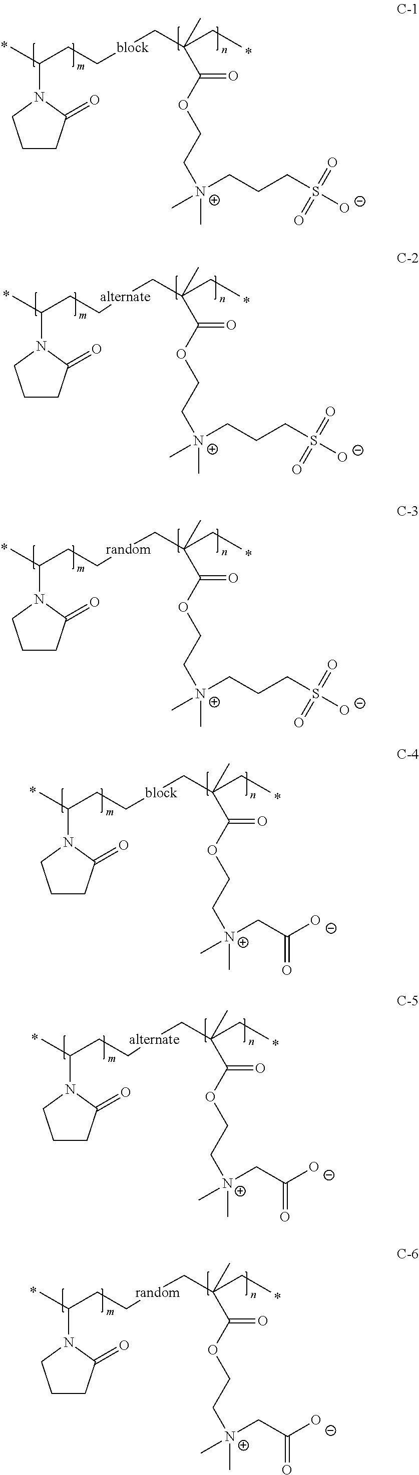

The biomimetic agents with formula (C-1)˜(C-6) can be a block, random or alternating copolymer comprising a cross-linkable moiety. After the biomimetic agents with formula (C-1)˜(C-6) is coated on a surface, the cross-linkable moiety can cross-link with each other by heating, Therefore, an antibiofouling network can be formed from the copolymer with formula (C-1)˜(C-6) by a thermal process. The thermal process is annealing process operated at 200° C.

In general, zwitterionic polymer, such as poly(sulfobetaine methacrylate) (PSBMA) exhibited the expected bio-inert nature, but the thermo-setting zwitterionic polymer (PSBMA or poly(carboxybetaine methacrylate) polymer networks showed the lost of fouling resistant control with the high-temperature annealing process. However, the present invention disclose that the modulated composition of thermo-setting poly(vinylpyrolidone)-co-poly(sulfobetaine methacrylate) copolymer networks having the specific mole mass ratio of poly(vinylpyrolidone) to poly(sulfobetaine methacrylate) effectively resist fibrinogen adsorption, platelet adhesion, erythrocytes attachment, bacteria attachment, cell attachment, hemolysis in 100% human plasma and 100% human whole blood. Accordingly, a method for preventing from fouling of a biomolecule on a substrate by forming a network on the substrate and an antibiofouling network are disclosed in the following embodiments of the invention.

In another embodiment of the invention, a method for preventing from fouling of a biomolecule on a substrate is provided. The method comprising applying a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate) to a substrate; and performing a thermal process at 120° C. above to have the copolymer to form a network on the substrate surface so as to prevent fouling on the substrate from a biomolecule. Preferably, the copolymer further comprises (Poly((octadecyl acrylate)-alt-((acrylic acid)-(N-(3-(dimethylamino)propyl) acrylamide))), poly(vinylpyrolidone)-block-poly(carboxybetaine methacrylate), poly(vinylpyrolidone)-random-poly(carboxybetaine methacrylate), and poly(vinylpyrolidone)-alternating-poly(carboxybetaine methacrylate).

In one example of another embodiment, the substrate is selected from the group consisting of metal, glass, wafer, polymer and ceramic. Preferably, the substrate is a metal which is selected from Fe and Fe alloys.

In one example of another embodiment, the copolymer has the mole mass ratio of poly(vinylpyrolidone) to poly(sulfobetaine methacrylate) being 68/32˜39/61

In one example of another embodiment, the network has the mole mass ratio of poly(vinylpyrolidone) to poly(sulfobetaine methacrylate) being 70/30˜42/58.

In one example of another embodiment, the thermal process is annealing process. Preferably, the annealing process is operated at 200° C. or above.

In one example of another embodiment, the network forming on all or part of a stent surface.

In one example of another embodiment, the network forming on all or part of surgical instruments surface which comprises a scalpel surface and an endoscope surface.

In one example of another embodiment, the network forming on all or part of a catheter surface.

In one example of another embodiment, the network forming on all or part of a lens surface which comprises intraocular lens surface.

In one example of another embodiment, the network forming on all or part of a blood separation device surface.

In one example of another embodiment, the network forming on forming on all or part of a marine device surface.

In one example of another embodiment, the network forming on forming on all or part of dental instruments surface and dentures surface which comprises dental implants surface.

In one example of another embodiment, the network forming on all or part of an artificial joint surface.

In one example of another embodiment, the biomolecule comprising fibrinogen, platelets, erythrocytes, fibroblast, and E. coli.

In still another embodiment of the invention, an antibiofouling network is disclosed. The antibiofouling network has the mole mass ratio of poly(vinylpyrolidone) to poly(sulfobetaine methacrylate) in a range of 70/30˜42/58 and is derived from a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate) by heating the copolymer at 120° C. above.

In one example of still another embodiment, the copolymer has the mole mass ratio of poly(vinylpyrolidone) to poly(sulfobetaine methacrylate) being 68/32˜39/61.

In one example of still another embodiment, the antibiofouling network being all or part of a stent.

In one example of still another embodiment, the antibiofouling network being all or part of surgical instruments which comprises a scalpel and an endoscopy.

In one example of still another embodiment, the antibiofouling network being all or part of a catheter.

In one example of still another embodiment, the antibiofouling network being all or part of a lens which comprises intraocular lens.

In one example of still another embodiment, the antibiofouling network being all or part of a blood separation device.

In one example of still another embodiment, the antifouling networks being all or part of a marine device.

In one example of still another embodiment, the antifouling networks being all or part of dental instruments and dentures which comprises dental implants.

In one example of still another embodiment, the antifouling networks being all or part of an artificial joint.

### Production Example 1

### Preparing A-33 (PODA-b-PSBMA)

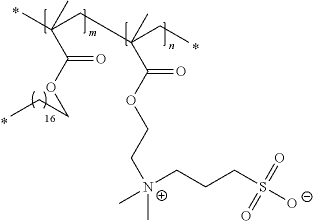

Production example 1: preparing A-33 (PODA-b-PSBMA Octadecyl acrylate (ODA) was dissolved in toluene to form a 40 wt % solution. By atomic transfer radical polymerization (ATRP), under existence of CuBr/2,2′-bipyridine (CuBr/bpy) as the catalyst and methyl 2-bromopropionate (MBrP) as the initiator, in nitrogen environment, [ODA]/[MBrP]/[CuBr]/[bpy](molar ratio) being 6/1/12˜120/1/1/2 was used to react for 24 hrs at 120° C. Then, by removing the catalyst and initiator, PODA was obtained.

By ATRP, poly(2-(dimethylamino)ethyl methacrylate) (DMAEMA) and PODA reacted with each other with a molar ratio of [DMAEMA]/[PODA]/[CuBr]/[bpy] being 5/1/1/2˜100/1/1/2 for 24 hrs at 120° C. to polymerize. Then, by removing the catalyst and initiator, the copolymer A-33P (Poly(octadecyl acrylate)-b-poly(2-(dimethylamino)ethyl methacrylate)) was obtained. 1,3-propanesultone and the copolymer A-33P were dissolved in THF to betainize the copolymer A-33P in atmospheric environment at room temperature for 24 hrs to obtain A-33.

### Production Example 2

### Preparing B-3 (Poly((octadecyl acrylate)-alt-((acrylic acid)-(N-(3-(dimethylamino)propyl) acrylamide))); Poly(ODA-alt-(AA-DMAPAMMI))

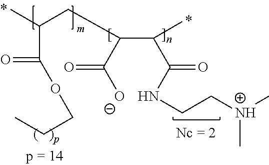

2 ml of 10 wt % PMAO (poly(maleic anhydride alt 1-octadecene)) solution (solvent is THF) was prepare. Dimethylethylenediamine (DMEA) 3.2 g was dissolved in THF (8 ml) to form a DMEA solution. PMAO solution was then added into DMEA solution to form precipitate. By centrifugal separation and extraction, the copolymer B-3 was obtained.

Other biomimetic agents for anti-biofouling coating in the above can be made or synthesized by similar method shown in the production examples 1 and 2 or modified method together with well-known polymerization methods. According to experiments conducted by the inventors, the biomimetic agents for anti-biofouling coating according to the invention show good resistance in adherence of biomolecules like protein (plasma protein, Fibrinogen, bovine serum albumin (BSA)), blood (such red blood, leukocyte, platelet), cell (human cell, Fibroblasts, Keratinocytes), bacteria (S. epidermidis, E. coli), etc. That is, less than 20% of biomolecules will adhere on the surface treated by the biomimetic agent for anti-biofouling coating according to the invention. Under the better condition, less than 2% of biomolecules will adhere on the treated surface. The invention also discloses a coating composition for anti-biofouling, comprising: the above mentioned biomimetic agent according to the invention and a solvent. The solvent can be, for example, water, phosphate-buffered saline, ethanol, methanol, etc. The biomimetic agent for anti-biofouling coating according to the invention can be applied to a surface such as polymeric surface like polytetrafluoroethylene (PTFE), polyvinylidene difluoride (PVDF), polystyrene (PS), polypropylene (PP), polyvinyl chloride (PVC) or inorganic oxide surface like SiO₂, Al₂O₃. In addition, a metallic surface like Ti, Fe can be treated by the biomimetic agent for anti-biofouling coating according to the invention.

### Example 3

### Preparation of poly(vinylpyrolidone)done) (PVP) and poly(vinylpyrolidone)-co-poly(sulfobetaine methacrylate) [poly(VP-co-SBMA)] copolymer gels and networks

The cross-linker N,N′-methylenebisacrylamide (MBAA) was dissolved in DI water at 1.6 wt % and stirred for 6 hr at 50° C. [2-(Methacryloyloxy)ethyl]-dimethyl-(3-sulfopropyl)-ammonium hydroxide (SBMA) and 1-vinyl-2-pyrrolidone (VP) of varying molar ratios were added to the MBAA solution, and the solution mixture was stirred for 10 min or until it was completely dissolved. The copolymeric reaction of the hydrogels was initiated by 0.2 wt % of Ammonium persulfate (APS) and 0.2 wt % of N,N,N′,N-tetraethylmethylenediamine (TEMED), and the reacting solution was then immediately transferred to a container to form a layer with the thickness of 0.2 mm. After polymerizing for 3 hr at 25° C., the gels were immersed in a water base for 48 hr to remove the chemical impurities. All the gels were made in disk-form with a diameter of 10.0 mm (10 mm biopsy punch, Acuderm Inc.) and stored in DI water at 4° C. till ready to use. The prepared gels were later dried in an evaporated oven to remove extra water to form a consistent shape. Finally, the polymer and copolymer networks were annealed at 200° C. to fix their shape with thermo-setting.

Characterizations of Poly(VP-co-SBMA) Copolymer Gels and Networks with Thermo-Setting Control.

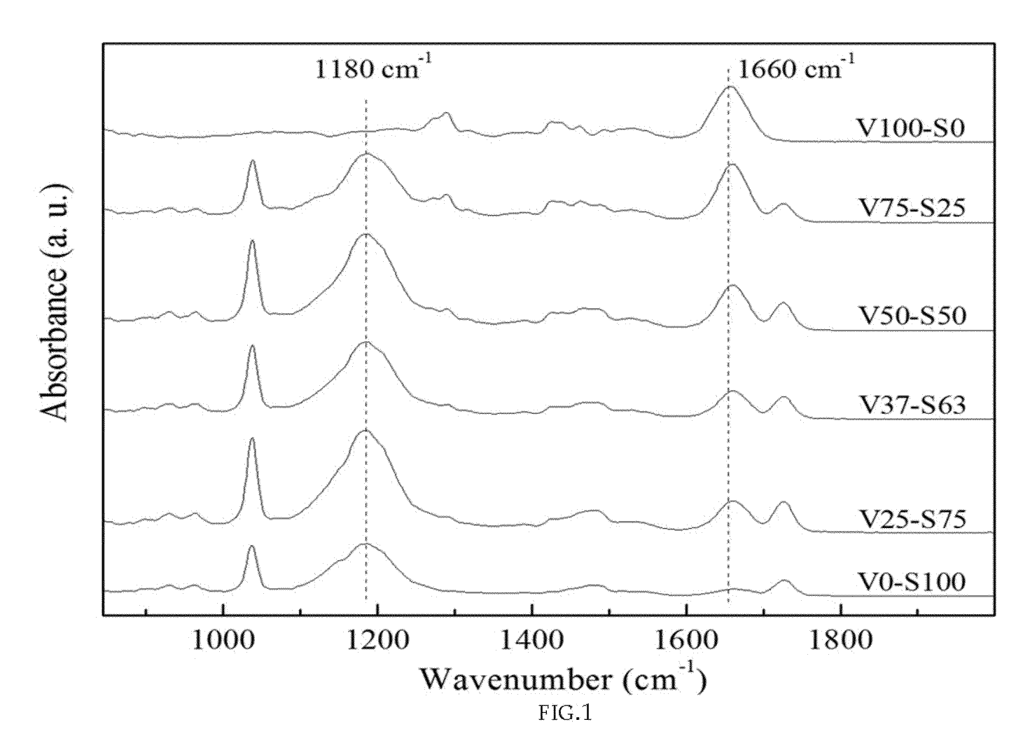

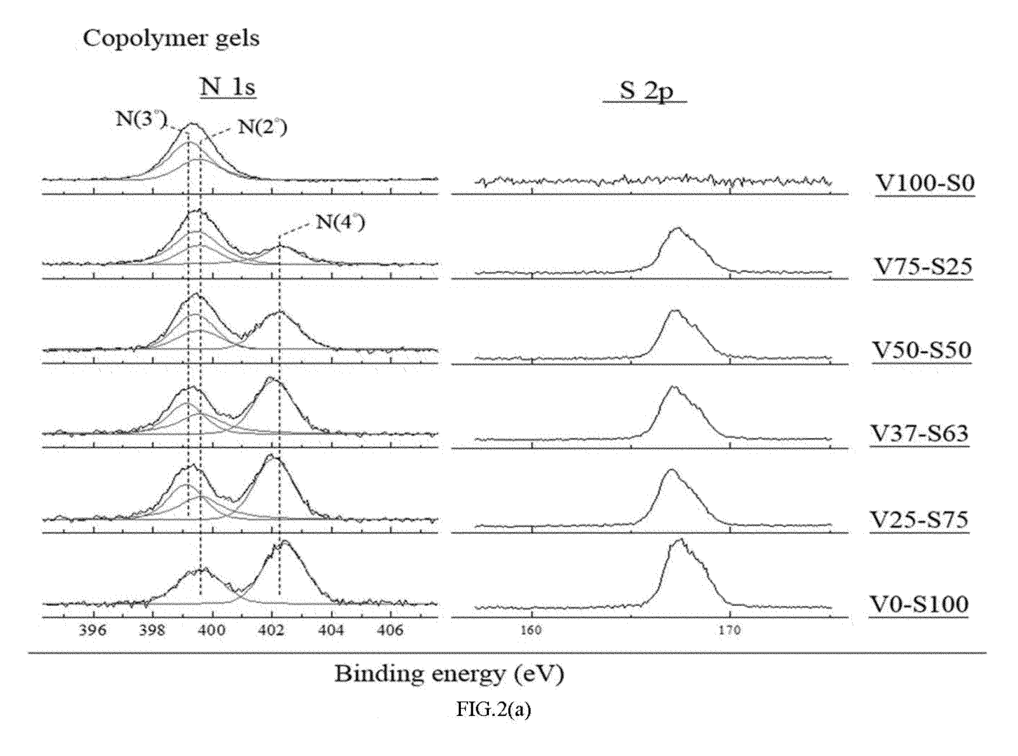

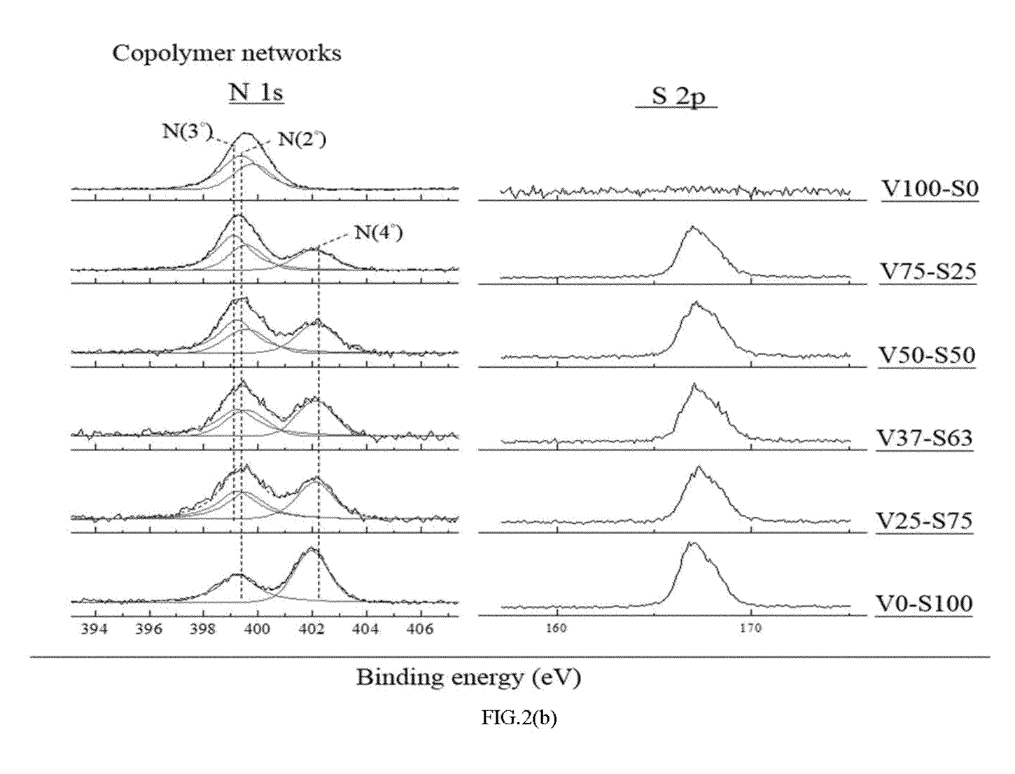

Chemical characterization of prepared poly(VP-co-SBMA) copolymer samples (Vm-Sn) are analyzed by Fourier transform infrared spectroscopy (FT-IR) and X-ray photoelectron spectroscopy (XPS). The FT-IR spectrums of PVP, poly(VP-co-SBMA), and PSBMA (co)polymer gels and networks were shown in FIG. 1. To achieve the quantitative identification of VP/SBMA composition, the SBMA mole fraction of poly(VP-coSBMA) copolymer gels and networks in the dry state was determined by XPS from the spectral area ratio of the atomic percentages based on the N 1s of the pyrrolidone group from PVP segments and S 2p of the sulfonate group from PSBMA segments at the BE of 399 and 168 eV, respectively. FIG. 2(a) to FIG. 2(b) show XPS spectrums of the VP and SBMA composition of N is and S 2p core-level spectra on the PVP (V100-S0), poly(VP-co-SBMA) (V75-S25, V50-S50, V37-S63, V25-S75), and PSBMA (V0-S100) gel and network interfaces determined with thermo-setting control, where Vm-Sn is poly(VP-coSBMA) derived from the molar ratio of m % of VP and n % of SBMA. For example, V75-S25 is poly(VP-co-SBMA) derived from the molar ratio of 75% of VP and 25% of SBMA. From the XPS analysis, the 4° N peak can be identified as positively charged groups of —N(CH₃)₂₊— and the S 2p peak can be identified as negatively charged groups of —SO₃— in the PSBMA segments. Thus, 4° N/S ratio is a good indicator to demonstrate the resulted charge neutrality of zwitterionic SBMA moieties distributed in the prepared copolymer networks associated with the thermo-setting control. Sample ID V100-S0 and V75-S25 was decomposed with thermo-setting. It was found that the poly(VP-co-SBMA) networks prepared with thermo-setting at high annealing temperature of 200° C. had the 4° N/S ratios of about 1.15±0.55 (1.05˜1.16), indicating a positive charge bias from zwitterionic neutrality. It might be due to the partial decomposition of the sulfonic acid groups (—SO₃—) at high temperature resulting in an unbalance charge-bias of the PSBMA segments. The analytical data was summarized in TABLE I.

TABLE I

| | molar | poly(VP-co-SBMA) gels | | | poly(VP-co-SBMA) networks | | |
|---|---|---|---|---|---|---|---|
| | ratio^{a} | without thermo-setting^{b} | | | with thermo-setting^{b} | | |
| | [VP]/ | (PVP)/ | swelling | Contact | (PVP)/ | swelling | contact |
| sample ID | [SBMA] | (PSBMA) | ratio | angle | (PSBMA) | ratio | angle |
| V100-S0 | 100/0 | 100/0 | 9.19 | 152 | — | — | — |
| V75- S25 | 75/25 | 81/19 | 3.01 | 153 | — | — | — |
| V50-S50 | 50/50 | 68/32 | 2.41 | 155 | 70/30 | 2.05 | 147 |
| V37- S63 | 37/63 | 39/61 | 2.26 | 159 | 42/58 | 1.94 | 143 |
| V25- S75 | 25/75 | 26/74 | 1.99 | 164 | 31/69 | 1.83 | 141 |
| V0-S100 | 0/100 | 0/100 | 1.62 | 165 | 0/100 | 1.47 | 139 |

^{a}Reaction molar ratio of co-monomers to initiator was fixed at 90 to 1. The total reactant mass percentage was 20 wt % in the prepared reaction solution.
^{b}The mole mass ratio of PVP to PSBMA in the poly(VP-co-SBMA) copolymer gels and networks was determined by XPS in the dry state from the spectral area ratio of the atomic percentages based on the N 1s of the pyrrolidone group from PVP segments and S 2p of the sulfonate group from PSBMA segments at the BE of 399 and 168 eV, respectively.

Human Plasma Protein Adsorption, Human Blood Platelets and whole blood cell Adhesion.

The adsorption of fibrinogen, a human plasma protein, was evaluated by the enzyme-linked immunosorbent assay (ELISA) to improve the sensitivity of the target proteins. Human blood platelets and whole blood cells attachments were tested using blood samples from healthy volunteers. The protocol for human protein adsorption and human blood cell attachment was followed by the procedure described in Jiang, S. Blood-Inert Surfaces via Ion-Pair Anchoring of Zwitterionic Copolymer Brushes in Human Whole Blood. Adv. Funct. Mater. 2013, 23, 1100-1110.

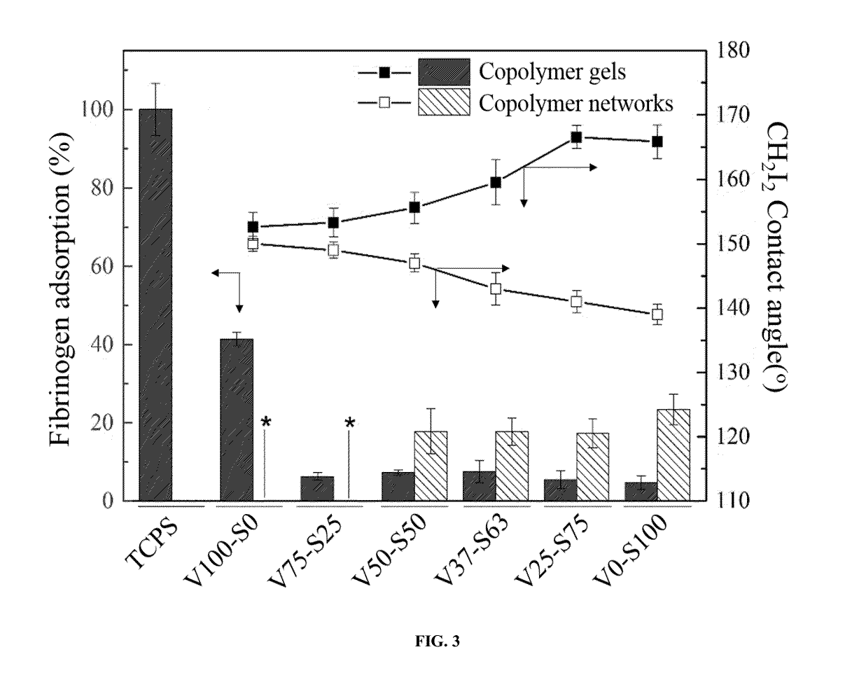

FIG. 3 shows the effect of SBMA composition in poly(VP-co-SBMA) copolymer gels on the correlation of diiodomethane contact angle related to protein adsorption with thermal treatment control. PSBMA gel surfaces with higher contact angle than that of PVP gel surfaces indicated better protein-resistant performance resulted from the more hydrophilicity of zwitterionic interfaces. The increased contact angle also indicated the enhancement in gel hydrophilicity of prepared poly(VP-co-SBMA) copolymer networks with the increase of the SBMA molar ratios. However, it was found that the contact angle of PSBMA networks (V0-S100) was reduced with the thermo-setting at 200° C., indicating an obvious decrease in gel interfacial hydrophilicity after the thermal treatment process. Thus, the observed protein adsorption on the V0-S100 surfaces at annealing temperature of 200° C. is over 20% higher than that on PSBMA networks without thermo-setting which might be due to the partial decomposition of the sulfonic acid groups (—SO₃—) at high temperature treatment resulting in an unbalance charge-bias of the PSBMA segments, as supported by XPS analysis. Most importantly, the increase of VP segments enhanced the protein resistance of poly(VP-coSBMA) networks with a much lower —SO₃— decomposition than that of PSBMA polymer networks. However, there is a composition limitation of VP segments in the poly(VP-co-SBMA) networks for the thermal tolerance with cracking, such as samples of V100-S0 and V75-S25. Herein, the composition of VP and SBMA segments in copolymer network with about 40 mol % VP in V37-S36 sample is modulated to obtain high fibrinogen resistance without thermal cracking and with controllable charge neutrality of zwitterionic nature.

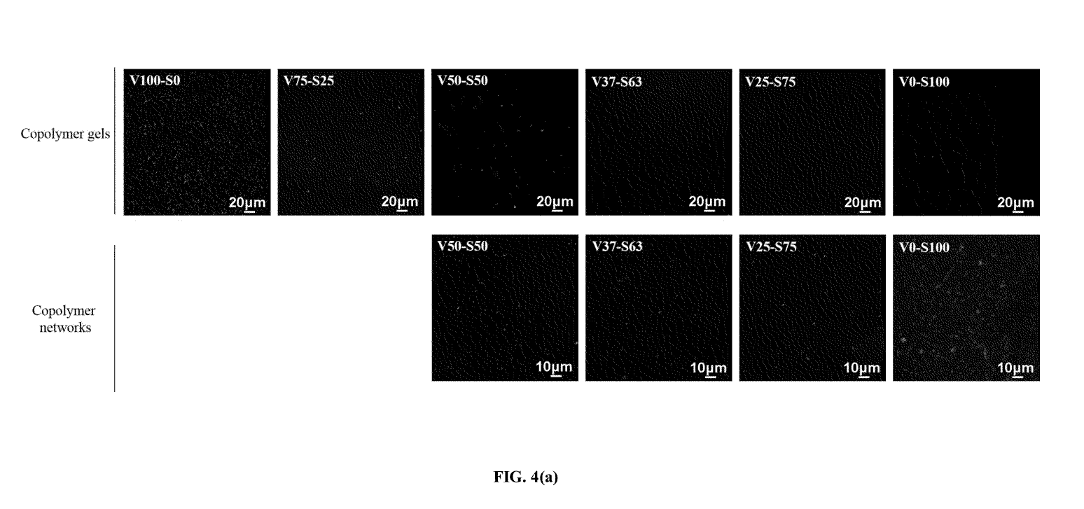

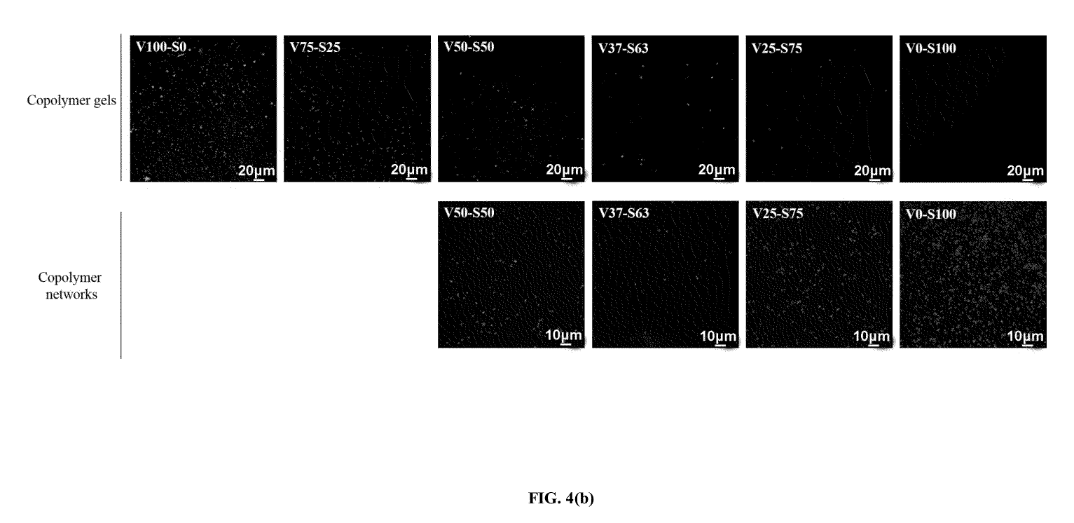

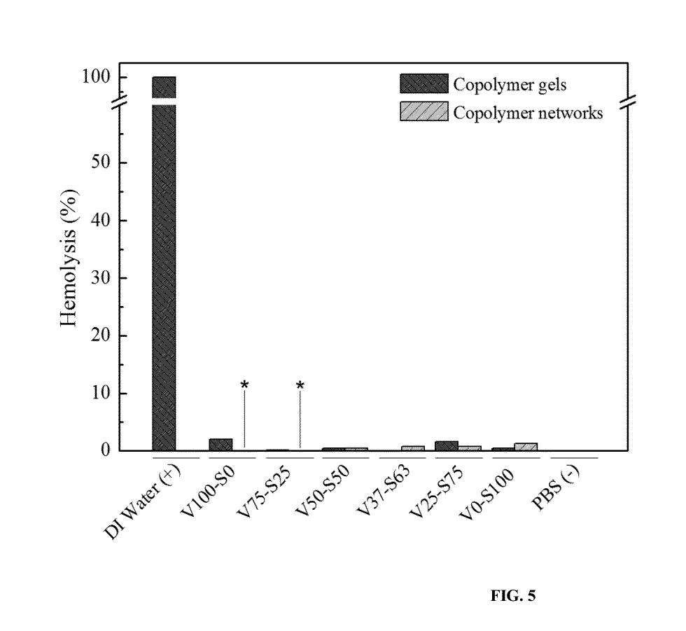

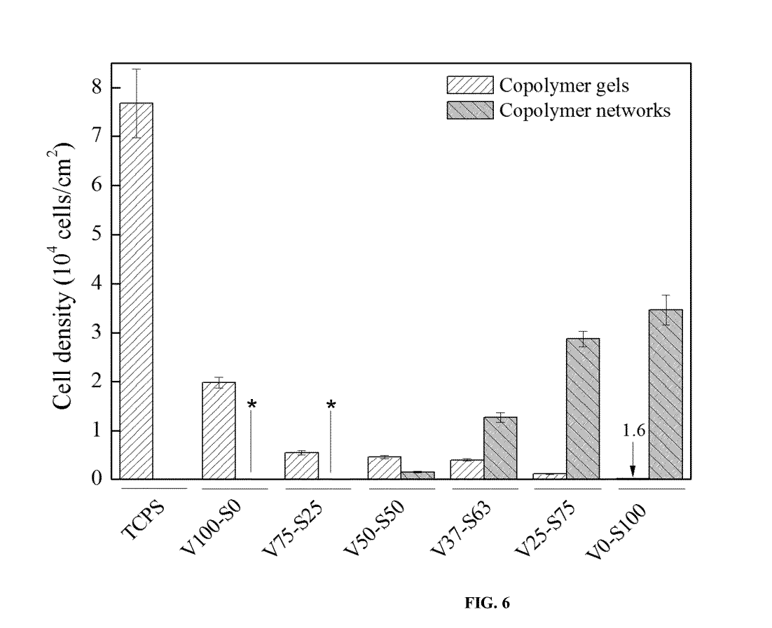

FIG. 4(a) to FIG. 4(b) showed a set of CLSM images for human platelet adhesion and erythrocyte attachment on the prepared copolymer gel and network surfaces at a magnification of 1000×. The prepared samples came into contact with platelet-rich plasma (PRP) solution, which was separated from human whole blood by centrifugation of 1200 rpm for 10 min at 37° C. It can be clearly observed in FIG. 6a that the platelets have almost no adhesion on the zwitterionic PSBMA gel surfaces compared to that on PVP gels without thermo-setting. Large number of platelet adhesion on the PVP gel surfaces is due to the activated interface with adsorbed fibrinogen. However, thermo-setting PSBMA network surfaces also lost the platelet resistance after the annealing process at 200° C. It was found that the platelet-resistant property for thermo-setting poly(VP-co-SBMA) network surfaces was well preserved, especially for the case of V37-S63. This observation strongly correlated to the fibrinogen adsorption in FIG. 5 which confirms the previous hypothesis that the surfaces with above a certain level of protein adsorption might lead to the adhesion and activation of platelets from human plasma. On the basis of protein adsorption and platelet adhesion described above, it was clear to see that poly(VP-co-SBMA) copolymer networks with a tunable molar ratio of zwitterionic SBMA segments can be used to achieve dual functions of high temperature tolerance shaping and good hemocompatibility in contact with human plasma solution.

Red Blood Cell Hemolysis.

The red blood cell (RBC) hemolysis assay was performed to evaluate the antihemolytic activity and blood compatibility of prepared copolymer gels and networks. When red blood cells encounter incompatible environments, such as a hydrophobic surface or a non-isotonic environment, the blood cell membrane might be destroyed and thus hemoglobin proteins are released out from the disrupted membranes of blood cells. By detecting the absorbance of hemoglobin at 541 nm, it is easy to identify the amount of released hemoglobin and clarify the hemolysis level. DI water (+) and PBS (−) were set as positive and negative controls for the hemolysis of RBCs, as shown in FIG. 5. All of the poly(VP-co-SBMA) copolymer gels and networks with thermo-setting controls kept of hemolysis at a safe level of 2% or lower, indicating good antihemolytic activity of copolymer interface in contact with blood solutions.

Cell Attachment Assay.

Human HT-1080 fibroblasts (ATCC, Manassas, Va.) modified with stable luciferase and EGFP expression by using the plasmid pAAVluciferase-EGFP were used to examine the attachment behavior of cells on the polymeric gel surfaces. The cells were cultured in Dulbecco's Modified Eagle Medium (DMEM) supplemented with 10% fetal bovine serum, 100 U/mL penicillin, and 100 μg/mL streptomycin at 37° C. in a humidified atmosphere with 5% CO₂. A cell suspension at 2×10⁴ cells/mL prepared, and 1 mL the cell suspension was allocated onto wither the copolymer gels or network disks, which were then incubated for three days at 37° C./5% CO₂. A Nikon TS 100 microscope with a 10× objective lens and a blue excitation fluorescence (450-490 nm) filter was used to observe the cells after proliferation and the fluorescence images were used to quantify the amount of cell attachment on the surfaces of the six independent disks (n=6 in total) for each gel substrate, and the average result was reported.

Fibroblast Cell Attachment on Poly(VP-co-SBMA) Copolymer Gels and Networks.

FIG. 6 showed the quantitative results of human fibroblast (HT1080) adhesion using the standard assay cultured on the poly(VP-co-SBMA) copolymer gel and network surfaces, and compared to the TCPS positive control surfaces. For the prepared copolymer gels without thermo-setting, the decreased HT1080 cells attachment was observed with increased amount of PSBMA segments in poly(VP-co-SBMA) copolymer gels. There are no attached cells observed on the zwitterionic PSBMA polymer gel surfaces. However, the thermo-setting PSBMA network surfaces showed different results in cells attachment. It was also observed that there was a good cell adhesive resistance as well as blood-inert property for the thermo-settable poly(VP-co-SBMA) copolymer networks with a specified control of overall charge neutrality of PSBMA segments modulated by the certain amount of PVP composition.

Bacteria Adhesion Assay.

Escherichia coli (E. coli), was used to investigate bacterial adhesion behavior on the surface of poly(VP-co-SBMA) copolymer gels without thermo-setting and copolymer networks with thermo-setting. The cultures of E. coli were incubated in the medium (3.0 mg/mL beef extract and 5.0 mg/mL peptone) at 37° C. and shaken at 100 rpm for 12 hr to reach the stationary phase then plated on either the prepared copolymer gel or network at concentration of 10⁶ cells/mL. The samples were cleaned before use by submerging the prepared copolymer gels or networks in 75 wt % ethanol for 1 hr and washed by PBS solution for three times. Each gel was then put into the well a 24 well plate, to which 1 mL of bacteria suspension was added. The samples were then incubated in an oven for 24 hr at 37° C. After incubation, the bacterial suspensions were removed from the wells the all samples were washed with PBS for three times to remove the bacteria that had accumulated but not adhered on the surfaces. Live/Dead Baclight was added to the samples to stain the adhering bacteria and the stained samples were washed with PBS three times then observed with the fluorescence microscope using 450-490 nm excitation, an Olympus BX51 CCD camera and a 10× objective lens.

Antibacterial Efficacy of Poly(VP-co-SBMA) Copolymer Gels and Networks.

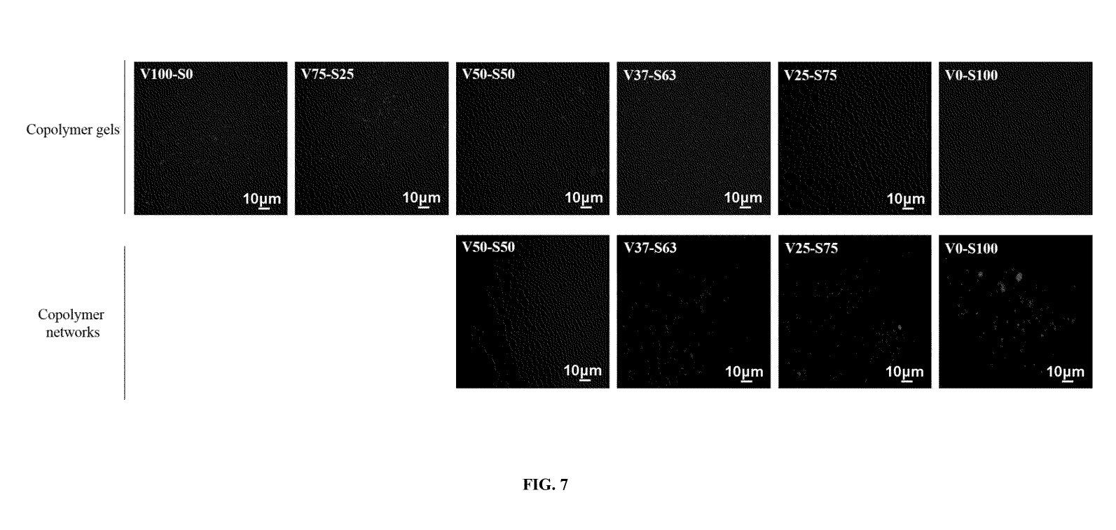

FIG. 7 showed a set of qualitative images of attached E. coli on poly(VP-co-SBMA) copolymer gels and networks. Live/Dead BacLight assay was used to characterize the long term accumulation of the E. coli on the prepared sample surfaces for 24 h at 37° C. It was clear that the bacteria were adhered onto the PVP polymer gel surfaces. The increased attachments of E. coli on poly(VP-co-SBMA) copolymer gel surfaces were also observed with the increase in mass composition of PSBMA segments in the copolymer gels without thermo-setting. Zwitterionic PSBMA gel surfaces showed perfect resistance of bacterial adhesion as well as protein adsorption, blood cell attachment, and tissue cell adhesion. Most importantly, the observed bacteria on these gel surfaces are all active, indicating non-toxicity of PVP or PSBMA segments for the accumulated E. coli. However, it was found that there is the dead bacterial adhesion with red color observed on the thermo-setting PSBMA polymer networks. The result supported the idea that a part of —SO₃⁻ decomposition in sulfobetaine groups at high temperature resulting in a positively charge-bias of the PSBMA segments by XPS analysis in FIG. 2. Thus, the zwitterionic pendent groups of the sulfobetaine (—CH₂CH₂N⁺(CH₃)₂CH₂CH₂CH₂SO₃⁺) partially converted to the positively charged groups containing quaternary ammonium (—CH₂CH₂N⁺((CH₃)₂—) in the PSBMA segments. In general, cationic surface can kill approaching bacteria or inhibit their growth on the material substrates. As the PVP segments in the thermo-setting poly(VP-co-SBMA) copolymer networks increased, the result is the thermal tolerance of zwitterionic sulfobetaine pendent group at high annealing temperature. It is supported by the observed results of no life or dead E. coli attached on the network surfaces of thermo-setting V50-S50 and V37-S63. The results also support the attachment of E. coli correlated with the protein adsorption and cell adhesion on the material interfaces of poly(VP-co-SBMA) copolymer gels and networks.

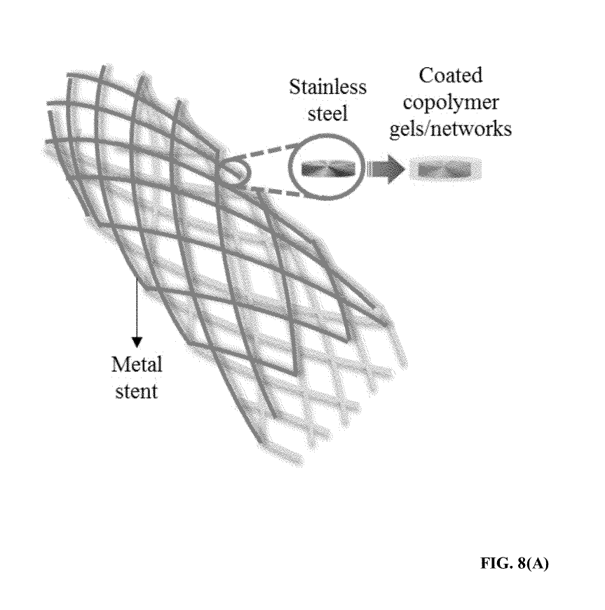

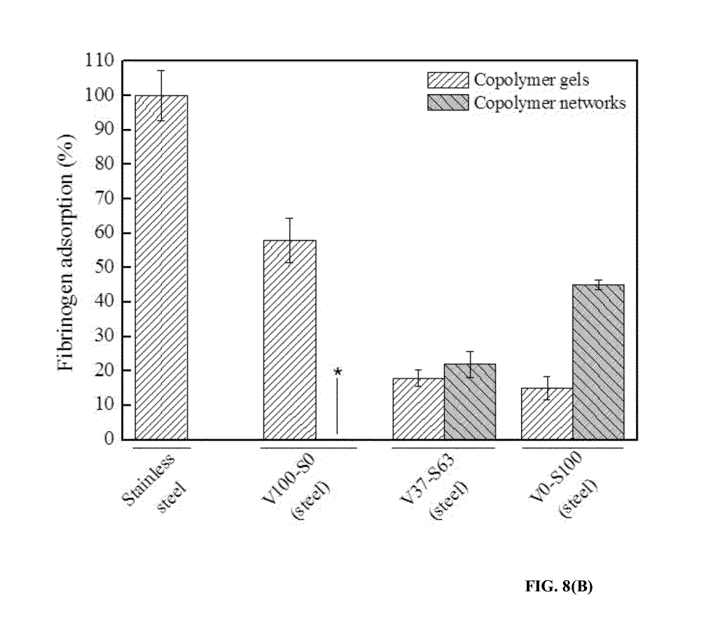

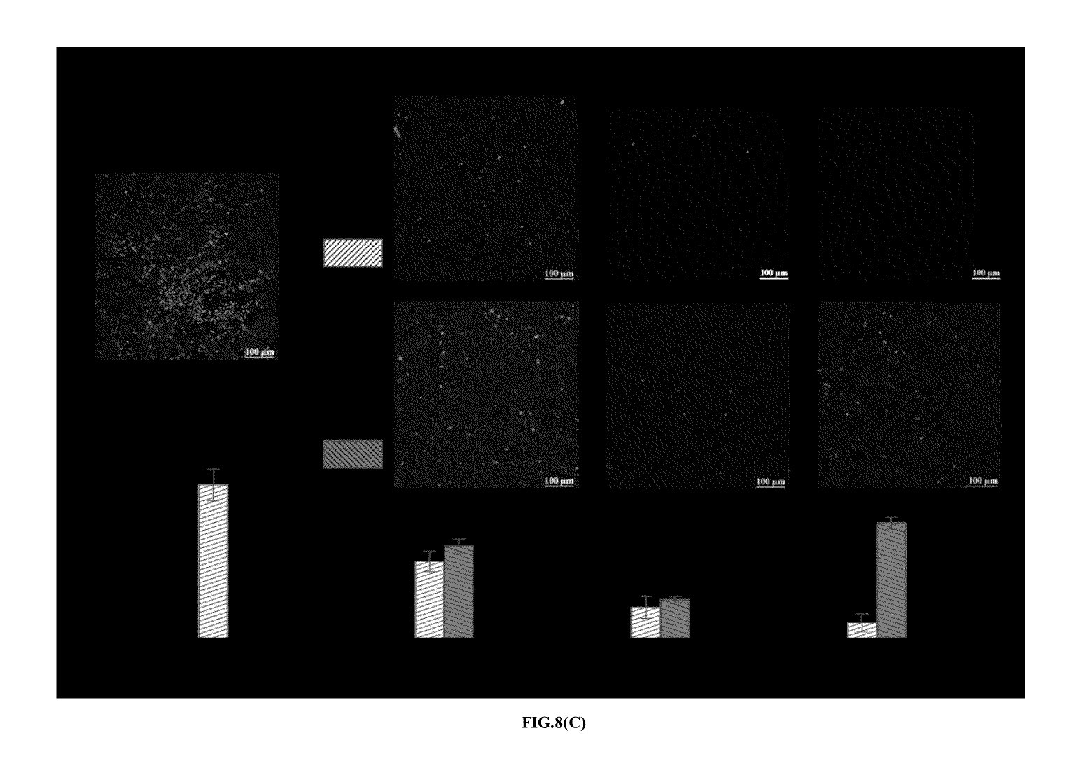

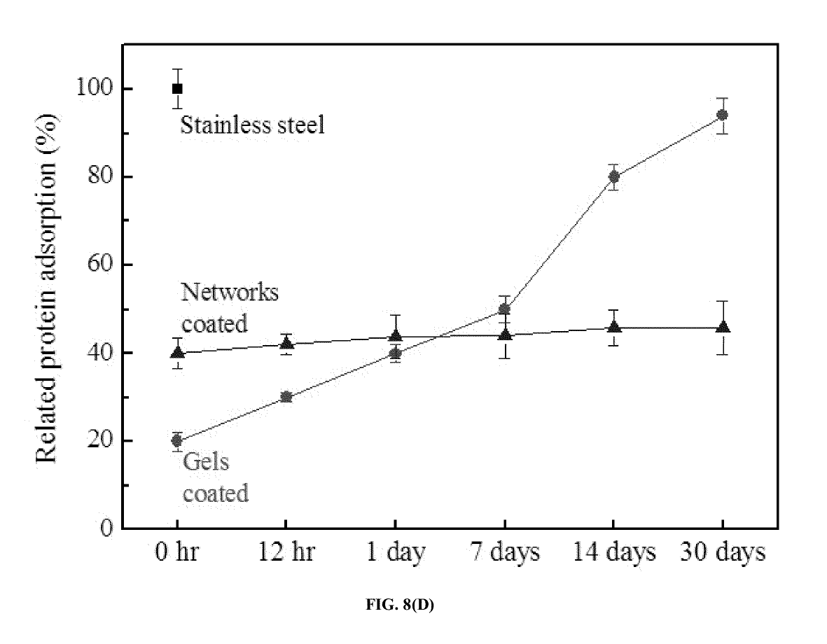

According to FIG. 8, application of poly(VP-co-SBMA) networks on the metal stent was shown in FIG. 8 (A). The result of fibrinogen adsorption test of copolymer gels/networks coated stainless steel disk was shown in FIG. 8 (B). The result of whole blood cell attachment test of copolymer gels/networks coated stainless steel disk was show in FIG. 8 (C). The result of stability of copolymer gels and networks coated on stainless steel disks in 30 days was show in FIG. 8 (D). All of the results indicate that the network derived from V37-S63 has excellent antibiofouling effects and long time storage stability.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

1. A method for preventing from fouling of a biomolecule on a substrate, said method comprising: applying a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate), to a substrate; and performing a thermal process at 120° C. above to have the copolymer to form a network on the substrate surface so as to prevent from fouling of a biomolecule on the substrate.

2. The method according to claim 1, wherein the copolymer has the mole mass ratio of poly(vinylpyrolidone) to poly(sulfobetaine methacrylate) being 68/32˜39/61.

3. The method according to claim 1, wherein the copolymer further comprises (Poly((octadecyl acrylate)-alt-((acrylic acid)-(N-(3-(dimethylamino)propyl) acrylamide))), poly(vinylpyrolidone)-block-poly(carboxybetaine methacrylate), poly(vinylpyrolidone)-random-poly(carboxybetaine methacrylate), and poly(vinylpyrolidone)-alternating-poly(carboxybetaine methacrylate).

4. The method according to claim 1, wherein the substrate is selected from the group consisting of metal, glass, wafer, polymer and ceramic.

5. The method according to claim 4, wherein the metal is Fe and Fe alloys.

6. The method according to claim 1, wherein the network has the mole mass ratio of poly(vinylpyrolidone) to poly(sulfobetaine methacrylate) being 70/30˜42/58.

7. The method according to claim 1, wherein the thermal process is annealing process.

8. The method according to claim 1, wherein the network forming on all or part of a stent surface.

9. The method according to claim 1, wherein the network forming on all or part of surgical instruments surface which comprises a scalpel surface and an endoscope surface.

10. The method according to claim 1, wherein the network forming on all or part of a catheter surface.

11. The method according to claim 1, wherein the network forming on all or part of a lens surface which comprises an intraocular lens surface.

12. The method according to claim 1, wherein the network forming on all or part of a blood separation device surface.

13. The method according to claim 1, wherein the network forming on all or part of a marine device surface.

14. The method according to claim 1, wherein the network forming on all or part of dental instruments surface and dentures surface which comprises dental implants surface.

15. The method according to claim 1, wherein the network forming on all or part of an artificial joint surface.

16. The method according to claim 1, wherein the biomolecule comprising fibrinogen, platelets, erythrocytes, fibroblast, and E. coli.

17. An antibiofouling network, said antibiofouling network having a mole mass ratio of poly(vinylpyrolidone) to poly(sulfobetaine methacrylate) in a range of 70/30˜42/58 and being derived from a copolymer comprises poly(vinylpyrolidone)-block-poly(sulfobetaine methacrylate), poly(vinylpyrolidone)-random-poly(sulfobetaine methacrylate) and poly(vinylpyrolidone)-alternating-poly(sulfobetaine methacrylate).

18. The antibiofouling network according to claim 17, wherein the copolymer has the mole mass ratio of poly(vinylpyrolidone) to poly(sulfobetaine methacrylate) being 68/32˜39/61.

19. The antibiofouling network according to claim 17, being all or part of a stent.

20. The antibiofouling network according to claim 17, being all or part of surgical instruments which comprises a scalpel and an endoscope.

21. The antibiofouling network according to claim 17, being all or part of a catheter.

22. The antibiofouling network according to claim 17, being all or part of a lens which comprises an intraocular lens.

23. The antibiofouling network according to claim 17, being all or part of a blood separation device.

24. The antifouling network according to claim 17, being all or part of a marine device.

25. The antifouling network according to claim 17, being all or part of dental instruments and dentures which comprises dental implants.

26. The antifouling network according to claim 17, being all or part of an artificial joint.
